(19) United States
(12) Patent Application Publication
(10) Pub. No.: US 2026/0294211 A1
(43) Pub. Date: Oct. 1, 2026

(54) ULTRASONIC ENDOSCOPE

(71) Applicant: FUJIFILM Corporation, Tokyo (JP)

(72) Inventors: Hirotoshi KOSAWA, Ashigarakami-gun (JP); Nori MIYAGISHIMA, Ashigarakami-gun (JP)

(73) Assignee: FUJIFILM Corporation, Tokyo (JP)

(21) Appl. No.: 19/572,911

(22) Filed: Mar. 20, 2026

(30) Foreign Application Priority Data
Mar. 25, 2025 (JP) 2025-049687

(51) Int. Cl. A61B 1/00; A61B 1/06; A61B 8/00; A61B 8/12

(52) U.S. Cl. CPC A61B 1/00082; A61B 1/0676; A61B 8/12; A61B 8/445

(57) ABSTRACT

An ultrasonic endoscope includes: an insertion part that is to be inserted into a subject; plural oscillators that are disposed on a distal end side of the insertion part in a circumferential direction of the insertion part and that transmit and receive ultrasonic waves; a balloon that is capable of covering the plural oscillators; a first groove that is provided on a proximal end side of the plural oscillators and that is capable of retaining the balloon; and a second groove that is provided on a distal end side of the plural oscillators and that is capable of retaining the balloon, and a length from the first groove to a distal end of the insertion part is longer than a length of the balloon in an axial direction.

### CROSS-REFERENCE TO RELATED APPLICATION

The present application claims priority under 35 U.S.C. § 119 to Japanese Patent Application No. 2025-049687 filed on Mar. 25, 2025. The above application is hereby expressly incorporated by reference, in its entirety, into the present application.

### BACKGROUND OF THE INVENTION

1. Field of the Invention
The technology of the present disclosure relates to an ultrasonic endoscope.
2. Description of the Related Art
WO2020/44905A discloses an ultrasonic endoscope in which a balloon is attachable and detachable.

### SUMMARY OF THE INVENTION

An object of the present disclosure is to provide an ultrasonic endoscope that can prevent a balloon from falling off.

An aspect of the technology of the present disclosure relates to an ultrasonic endoscope comprising: an insertion part that is inserted into a subject; a plurality of oscillators that are disposed on a distal end side of the insertion part in a circumferential direction of the insertion part and that transmit and receive ultrasonic waves; a balloon that is capable of covering the plurality of oscillators; a first groove that is provided on a proximal end side of the plurality of oscillators and that is capable of retaining the balloon; and a second groove that is provided on a distal end side of the plurality of oscillators and that is capable of retaining the balloon, in which a length from the first groove to a distal end of the insertion part is longer than a length of the balloon in an axial direction.

Another aspect of the technology of the present disclosure relates to an ultrasonic endoscope comprising: an insertion part that is inserted into a subject; a plurality of oscillators that are disposed on a distal end side of the insertion part in a circumferential direction of the insertion part and that transmit and receive ultrasonic waves; a balloon that is capable of covering the plurality of oscillators; a first groove that is provided on a proximal end side of the plurality of oscillators and that is capable of retaining the balloon; and a second groove that is provided on a distal end side of the plurality of oscillators and that is capable of retaining the balloon, in which a relationship between a cross-sectional diameter x of an end part of the balloon on a side to be retained in the second groove, a thickness t of an inflatable part of the balloon, and a width w and a depth h of the second groove satisfies at least one of w≥x+3t or h≥0.5x+2t.

According to the technology of the present disclosure, it is possible to provide the ultrasonic endoscope that can prevent the balloon from falling off.

### BRIEF DESCRIPTION OF THE DRAWINGS

FIG. 1 is a schematic configuration diagram illustrating an ultrasonic endoscope system 10 using an ultrasonic endoscope 12 that is an embodiment of an ultrasonic endoscope according to the present disclosure.

FIG. 2 is a partially enlarged perspective view illustrating an appearance of an example of a distal end part of the ultrasonic endoscope 12 illustrated in FIG. 1.

FIG. 3 is a cross-sectional view taken along an axis of the distal end part of the ultrasonic endoscope 12 illustrated in FIG. 2.

FIG. 4 is a side view of a balloon 60 attachable to a distal end part 40.

FIG. 5 is a cross-sectional view of the balloon 60 illustrated in FIG. 4 in a cross section passing through an axis of the balloon 60.

FIG. 6 is a view of the distal end part 40 as viewed in a radial direction.

FIG. 7 is a schematic diagram illustrating a process of attaching the balloon 60 to the distal end part 40.

FIG. 8 is a schematic diagram illustrating a process of attaching the balloon 60 longer than the example illustrated in FIG. 7 to the distal end part 40.

FIG. 9 is a diagram illustrating a state in which a bead part 62 in which an inflatable part 63 is wound one and a half turns is retained by a distal end groove 2.

### DESCRIPTION OF THE PREFERRED EMBODIMENTS

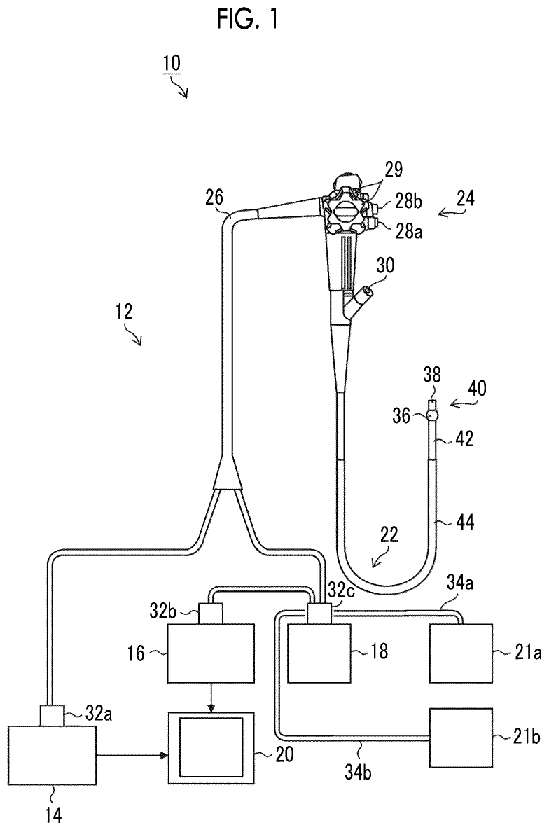

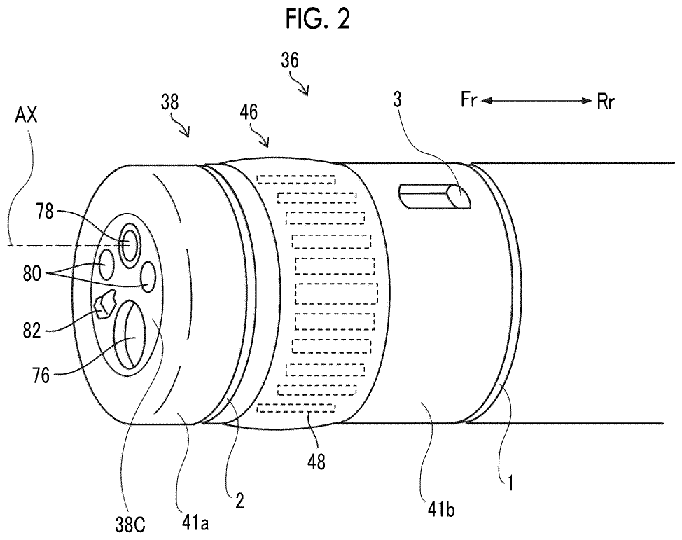

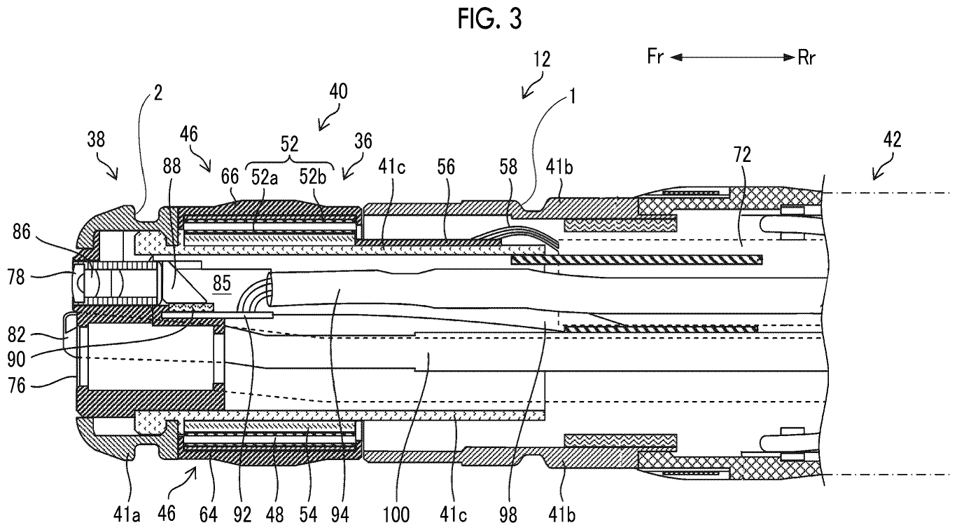

FIG. 1 is a schematic configuration diagram illustrating an ultrasonic endoscope system 10 using an ultrasonic endoscope 12 that is an embodiment of an ultrasonic endoscope according to the present disclosure. FIG. 2 is a partially enlarged perspective view illustrating an appearance of an example of a distal end part of the ultrasonic endoscope 12 illustrated in FIG. 1. FIG. 3 is a cross-sectional view taken along an axis of the distal end part of the ultrasonic endoscope 12 illustrated in FIG. 2.

As illustrated in FIG. 1, the ultrasonic endoscope system 10 comprises an ultrasonic endoscope 12, an ultrasonic processor device 14 that generates an ultrasound image, an endoscopic processor device 16 that generates an endoscopic image, a light source device 18 that supplies illumination light for illuminating an inner portion of a subject to the ultrasonic endoscope 12, a monitor 20 that displays the ultrasound image, the endoscopic image, and the like, a water supply tank 21a that stores washing water and the like, and a suction pump 21b that suctions an object to be suctioned in a body cavity.

The ultrasonic endoscope 12 has an insertion part 22 that is inserted into the body cavity of the subject, an operating part 24 that is consecutively provided in a proximal end part of the insertion part 22 and is used by an operator to perform an operation, and a universal cord 26 having one end connected to the operating part 24.

In the operating part 24, an air supply/water supply button 28a that opens and closes an air and water feeding conduit (not illustrated) from the water supply tank 21a, and a suction button 28b that opens and closes a suction conduit (not illustrated) from the suction pump 21b are provided side by side. Further, in the operating part 24, a pair of angle knobs 29 and a treatment tool insertion port 30 are provided.

An ultrasound connector 32a that is connected to the ultrasonic processor device 14, an endoscopic connector 32b that is connected to the endoscopic processor device 16, and a light source connector 32c that is connected to the light source device 18 are provided at the other end part of the universal cord 26. The ultrasonic endoscope 12 is attachably and detachably connected to the ultrasonic processor device 14, the endoscopic processor device 16, and the light source device 18 via the connectors 32a, 32b, and 32c, respectively. The connector 32c comprises an air supply/water supply tube 34a that is connected to the water supply tank 21a, and a suction tube 34b that is connected to the suction pump 21b.

The insertion part 22 includes, in order from a distal end side, a distal end part 40 formed of a hard member and having an ultrasonic observation part 36 and an endoscopic observation part 38, a bendable part 42 that is consecutively provided on a proximal end side of the distal end part 40, and a soft part 44 that connects a proximal end side of the bendable part 42 and a distal end side of the operating part 24. The bendable part 42 is formed by connecting a plurality of bendable pieces (angle rings) and is configured to be bendable. The soft part 44 is elongated, long, and flexible.

The ultrasonic processor device 14 generates and supplies an ultrasound signal for causing a plurality of oscillators 48 of an ultrasonic transducer 46 (see FIG. 2) of the ultrasonic observation part 36, which will be described later, to generate an ultrasonic wave. In addition, the ultrasonic processor device 14 receives and acquires an echo signal reflected from an observation target part irradiated with the ultrasonic wave, by the oscillator 48 and executes various kinds of signal processing on the acquired echo signal to generate an ultrasound image. The monitor 20 displays the generated ultrasound image.

The endoscopic processor device 16 receives and acquires an image signal acquired from the observation target part illuminated with illumination light from the light source device 18 in the endoscopic observation part 38, and executes various kinds of signal processing and image processing on the acquired image signal to generate an endoscopic image. The monitor 20 displays the generated endoscopic image.

In order to image the observation target part inside the body cavity using the endoscopic observation part 38 to acquire the image signal, the light source device 18 generates the illumination light, such as white light consisting of light of three primary colors of red light, green light, and blue light or light of a specific wavelength, and the illumination light propagates through a light guide (not illustrated) and the like in the ultrasonic endoscope 12 and is emitted from the endoscopic observation part 38, and the observation target part inside the body cavity is illuminated with the illumination light.

Hereinafter, a configuration of the distal end part 40 will be described with reference to FIGS. 2 and 3. FIG. 3 illustrates a distal end direction Fr from the proximal end side to the distal end side and a proximal end direction Rr from the distal end side to the proximal end side as a longitudinal direction of the insertion part 22 of the ultrasonic endoscope 12.

As illustrated in FIG. 2, the distal end part 40 is provided with the ultrasonic observation part 36 for acquiring the ultrasound image on the proximal end side and the endoscopic observation part 38 for acquiring the endoscopic image on the distal end side.

The distal end part 40 includes a cap-shaped distal end component 41a that covers the endoscopic observation part 38, a proximal end side ring 41b that is disposed on the proximal end side of the ultrasonic observation part 36, and a metal ring 41c (see FIG. 3) made of stainless steel (Steel Use Stainless (SUS)) or the like. The distal end component 41a and the proximal end side ring 41b consist of a rigid member such as a rigid resin and are exterior members. The metal ring 41c is disposed on an inner side of the exterior member.

The endoscopic observation part 38 includes a treatment tool outlet port 76, an observation window 78, an illumination window 80, a washing nozzle 82, and the like that are provided on a distal end surface 38C (see FIG. 2). The illumination window 80 emits the illumination light to the subject and is made of resin, glass, and the like. The observation window 78 is a window through which the reflected light of the illumination light is incident, and is configured as, for example, a concave lens. The distal end surface 38C is configured as a flat surface intersecting the longitudinal direction of the insertion part 22. In the example of FIG. 2, the distal end surface 38C is the flat surface perpendicular to the longitudinal direction of the insertion part 22. The flat surface is a substantially flat surface, and may include tolerances in practice. Since the distal end surface 38C is the flat surface, a range of light incident to the observation window 78 can be increased, and an imaging range of an observation system unit 85 can be expanded. Further, the illumination light from the illumination window 80 can reach a wide range.

The ultrasonic observation part 36 is configured by the ultrasonic transducer 46. The ultrasonic transducer 46 is configured by disposing the plurality of oscillators 48 that transmit and receive the ultrasonic wave in a circumferential direction of the insertion part 22.

The balloon 60 that can cover the ultrasonic transducer 46 is attachably and detachably provided on the distal end part 40. The balloon 60 is used to inject an ultrasound transmission medium (for example, water or oil) between the ultrasonic transducer 46 and the subject.

As illustrated in FIG. 3, in the distal end part 40, the observation system unit 85 is disposed rearward (on the proximal end side) of the observation window 78. The observation system unit 85 includes, for example, an objective lens 86, a prism 88, an imaging element 90, a substrate 92, and a signal cable 94.

The reflected light of the observation target part incident through the observation window 78 is captured by the objective lens 86. An optical path of the captured reflected light is bent at a right angle by the prism 88, and the reflected light is imaged on the imaging surface of the imaging element 90. The imaging element 90 photoelectrically converts the reflected light of the observation target part, which has transmitted through the observation window 78, the objective lens 86, and the prism 88 and imaged on the imaging surface, to output the image signal. An optical axis AX (same as the optical axis of the objective lens 86) of the observation window 78 extends in the longitudinal direction of the insertion part 22, and the ultrasonic endoscope 12 is configured as a so-called forward-viewing endoscope.

The imaging element 90 is mounted on the substrate 92. A circuit pattern (not illustrated) electrically connected to the imaging element 90 is formed on the substrate 92. The circuit pattern comprises a plurality of electrodes at an end part, and a plurality of signal cables 94 are connected to the plurality of electrodes. The plurality of signal cables 94 are connected to the endoscopic connector 32b (see FIG. 1). The endoscopic connector 32b is connected to the endoscopic processor device 16.

An emission end of a light guide 98 is connected to the illumination window 80 (see FIG. 2). An incidence end of the light guide 98 is connected to the light source device 18 via the universal cord 26. The illumination light emitted from the light source device 18 propagates through the light guide 98, and a part to be observed is irradiated with the illumination light from the illumination window 80.

In order to wash the surfaces of the observation window 78 and the illumination window 80, the washing nozzle 82 ejects air or washing water from the water supply tank 21a toward the observation window 78 and the illumination window 80 through an air supply/water supply channel 100 in the ultrasonic endoscope 12.

Various members such as a component constituting the endoscopic observation part 38, a conduit, and a transmission path extending from the proximal end side to the distal end side of the insertion part 22 are stored inside the metal ring 41c.

The ultrasonic transducer 46 constituting the ultrasonic observation part 36 includes the plurality of oscillators 48 (see FIG. 2) arranged in a cylindrical shape, an electrode part 52 that includes individual electrodes 52a corresponding to the plurality of oscillators 48 and a common electrode 52b common to the plurality of oscillators 48, a flexible printed substrate 56 to which each of the individual electrodes 52a is connected, and the metal ring 41c that supports the plurality of oscillators 48 wound around an outer periphery.

Since the flexible printed substrate 56 is thin and flexible, the flexible printed substrate 56 can be easily bent. A rigid substrate having high rigidity and having no flexibility can be used instead of the flexible printed substrate 56.

The ultrasonic transducer 46 further includes an acoustic matching layer 64 that is laminated on the oscillator 48 and an acoustic lens 66 that is laminated on the acoustic matching layer 64. The ultrasonic transducer 46 consists of a laminate of the acoustic lens 66, the acoustic matching layer 64, the oscillator 48, and the backing material layer 54. In addition, the laminate is bonded to an outer peripheral surface of the metal ring 41c by a method such as fitting.

As illustrated in FIG. 2, the oscillator 48 is an array of a plurality of channels, for example, 48 to 192 channels (CH), consisting of a plurality of rectangular parallelepiped-shaped oscillators 48 arranged in a cylindrical shape, for example, 48 to 192 oscillators 48. The oscillator 48 is disposed such that the longitudinal direction thereof matches the longitudinal direction of the insertion part 22.

In the ultrasonic transducer 46, the plurality of oscillators 48 are arranged at a predetermined pitch in the circumferential direction, as in the illustrated example. As described above, each oscillator 48 constituting the ultrasonic transducer 46 is arranged at equal intervals in a cylindrical shape around the longitudinal axis of the insertion part 22. Each oscillator 48 is sequentially driven based on a drive signal input from the ultrasonic processor device 14. As a result, radial electronic scanning is performed using, as a scanning range, a range in which the oscillators 48 are arranged.

As illustrated in FIG. 3, the flexible printed substrate 56 is electrically connected to each of the individual electrodes 52a of the electrode part 52 on one side and is wired and connected to a plurality of coaxial cables 58 of a signal line bundle 72 on the other side. In this way, the individual electrode 52a of the oscillator 48 and the coaxial cable 58 are electrically connected to each other, and each oscillator 48 and the signal line bundle 72 are electrically connected to each other.

As illustrated in FIG. 2, the distal end part 40 includes a proximal end groove 1 provided on the proximal end side of the ultrasonic transducer 46 and a distal end groove 2 provided on the distal end side of the ultrasonic transducer 46 on the outer peripheral surface thereof. The proximal end groove 1 constitutes a first groove. The distal end groove 2 constitutes a second groove. The proximal end groove 1 and the distal end groove 2 are formed over the entire circumference in the circumferential direction of the distal end part 40, and are configured to retain a tubular balloon 60. On the outer peripheral surface of the distal end part 40, a delivery port 3 that can deliver the ultrasound transmission medium is provided between the ultrasonic transducer 46 and the proximal end groove 1.

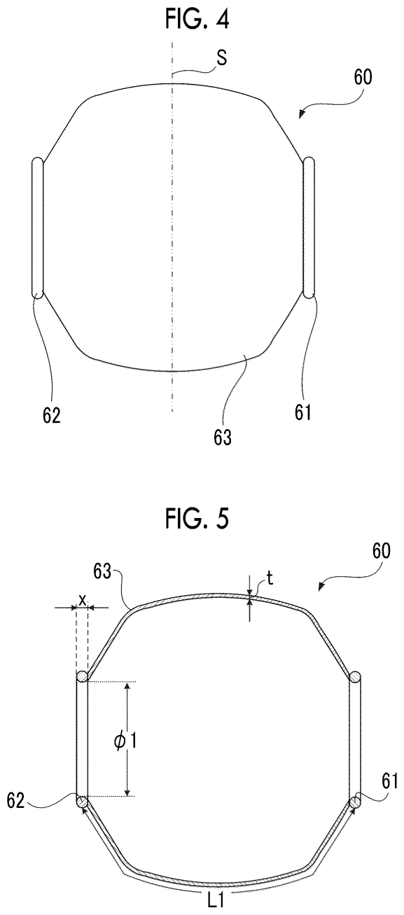

FIG. 4 is a side view of the balloon 60 attachable to the distal end part 40. FIG. 5 is a cross-sectional view of the balloon 60 illustrated in FIG. 4 in the cross section passing through the axis of the balloon 60.

The balloon 60 has a substantially cylindrical shape, the distal end part 40 is inserted into the balloon 60, and the balloon 60 is attached to the distal end part 40. The balloon 60 includes thick annular bead parts 61 and 62 provided at both ends in the axial direction and an inflatable part 63 between the bead parts 61 and 62.

The balloon 60 into which the distal end part 40 is inserted is attached to the distal end part 40 by the bead part 61 being retained by the proximal end groove 1 of the distal end part 40 and the bead part 62 being retained by the distal end groove 2 of the distal end part 40. The bead part 62 constitutes an end part on a side retained by the distal end groove 2 among both end parts of the balloon 60 in the axial direction.

The inflatable part 63 is configured to be thinner than the bead parts 61 and 62 and is configured to inflate in the radial direction in a case in which the ultrasound transmission medium is delivered from the delivery port 3 in a state in which the balloon 60 is attached to the distal end part 40.

As illustrated in FIG. 4, the balloon 60 has a shape that is line-symmetric about a straight line S that passes through the center in the axial direction and extends in the radial direction, but the present disclosure is not limited to this. In a case in which the balloon 60 has a line-symmetric shape, the balloon 60 can be attached to the distal end part 40 by retaining the bead part 61 by the distal end groove 2 and retaining the bead part 62 by the proximal end groove 1.

FIG. 5 illustrates an inner diameter φ1 of the bead part 62, a thickness t of the inflatable part 63, a cross-sectional diameter x of the bead part 62, and a length L1 of the balloon 60 in the axial direction, as information indicating a structure of the balloon 60.

The inner diameter φ1 means an inner diameter of a hole surrounded by the bead part 62 in a state in which the balloon 60 is made cylindrical and self-standing (a state in which the balloon 60 is placed with the axial direction of the balloon 60 as the horizontal direction, the same applies below). In a case in which the hole surrounded by the bead part 62 is not circular, the inner diameter φ1 is an equivalent circle diameter. This “equivalent circle diameter” means a diameter of a true circle corresponding to an area of the hole surrounded by the bead part 62.

In a case in which the cross section of the bead part 62 is circular, the cross-sectional diameter x means a diameter of the circle. In a case in which the cross section of the bead part 62 is not circular, the cross-sectional diameter x is an equivalent circle diameter. This “equivalent circle diameter” means a diameter of a true circle corresponding to an area of the cross section of the bead part 62.

The length L1 means a circumferential length of the balloon 60 in the axial direction, and indicates a length obtained by adding the cross-sectional diameter of the bead part 61 and the cross-sectional diameter of the bead part 62 to a circumferential length of the inflatable part 63 from the bead part 61 to the bead part 62.

The material of the balloon 60 is not particularly limited, but is preferably a synthetic elastomer or isoprene rubber. The synthetic elastomer is, for example, at least one of a styrene-based elastomer, an olefin-based elastomer, a vinyl chloride-based elastomer, a polyester-based elastomer, a polyurethane-based elastomer, a polyamide-based elastomer, a silicone-based elastomer, or a fluorine-based elastomer. By not including natural rubber in the material forming the balloon 60, it is possible to suppress the risk of developing latex allergy caused by natural rubber.

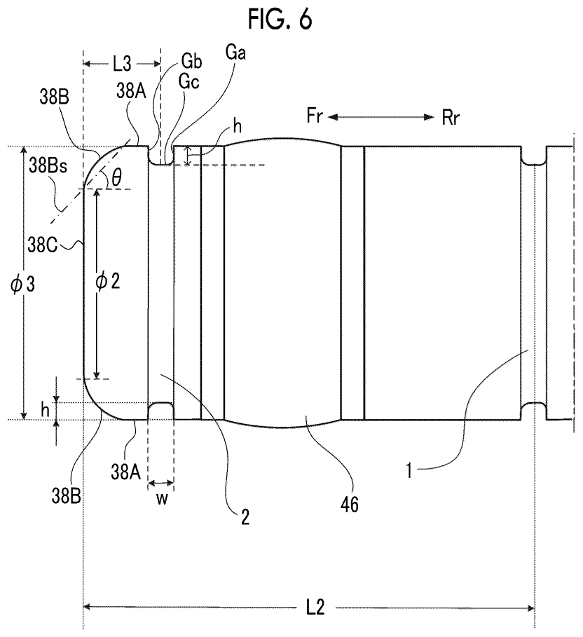

FIG. 6 is a view of the distal end part 40 as viewed in the radial direction (a direction orthogonal to the longitudinal direction of the insertion part 22) of the insertion part 22. FIG. 6 illustrates a width w and a depth h of the distal end groove 2. The distal end groove 2 includes an inner wall surface Ga on the proximal end side, an inner wall surface Gb on the distal end side, and a bottom surface Gc connecting the inner wall surfaces Ga and Gb. The two inner wall surfaces Ga and Gb are, for example, perpendicular to the longitudinal direction of the insertion part 22, and are parallel to each other. The width w indicates a distance between these two inner wall surfaces Ga and Gb.

As illustrated in FIG. 6, an outer peripheral surface of the distal end part 40 on the distal end side of the ultrasonic transducer 46 includes the distal end surface 38C, a side surface 38A (a surface perpendicular to the radial direction in the example of FIG. 6) that is located on the proximal end side of the distal end surface 38C and on the radially outer side of the insertion part 22 with respect to the distal end surface 38C and that intersects the radial direction of the insertion part 22, and a curved surface 38B that connects the side surface 38A and the distal end surface 38C.

In the side view of FIG. 6, in a case in which an imaginary straight line 38Bs connecting an end of the curved surface 38B on the distal end surface 38C side and an end of the curved surface 38B on the side surface 38A side is drawn, an angle θ formed between the imaginary straight line 38Bs and the longitudinal direction of the insertion part 22 is preferably equal to or less than 45 degrees. The curved surface 38B may be a flat surface (a surface that matches the imaginary straight line 38Bs in the side view) inclined at an angle of equal to or less than 45 degrees with respect to the longitudinal direction.

By making the angle θ equal to or less than 45 degrees, a path from the distal end surface 38C to the side surface 38A can be made steep. By making the path steep, in a case in which the bead part 62 moves along the path, the bead part 62 can be moved to the distal end groove 2 side with a small force, and the inflatable part 63 can be prevented from being wound around the bead part 62.

The distal end groove 2 is formed on the side surface 38A. Therefore, a diameter of a circle passing through the center of the distal end groove 2 is greater than an outer diameter φ2 of the distal end surface 38C. The outer diameter φ2 means a diameter of a circle in a case in which a shape of the distal end surface 38C as viewed in the longitudinal direction is circular. In a case in which the distal end surface 38C is not circular, the outer diameter φ2 is an equivalent circle diameter. This “equivalent circle diameter” means a diameter of a true circle corresponding to an area of the distal end surface 38C. The outer diameter φ2 is preferably less than the inner diameter φ1 of the opening surrounded by the bead part 62 of the balloon 60.

FIG. 6 illustrates an outer diameter φ3 of a portion of the insertion part 22 on the distal end side of the distal end groove 2. The outer diameter φ3 means a maximum value of a length of the portion in the radial direction. The outer diameter φ3 is preferably greater than the inner diameter φ1 of the opening surrounded by the bead part 62 of the balloon 60 and is equal to or less than twice the inner diameter φ1.

FIG. 6 illustrates a length L2 from the proximal end groove 1 (the center in the longitudinal direction) to the distal end surface 38C, which is the distal end of the insertion part 22. The length L2 is longer than the length L1 of the balloon 60.

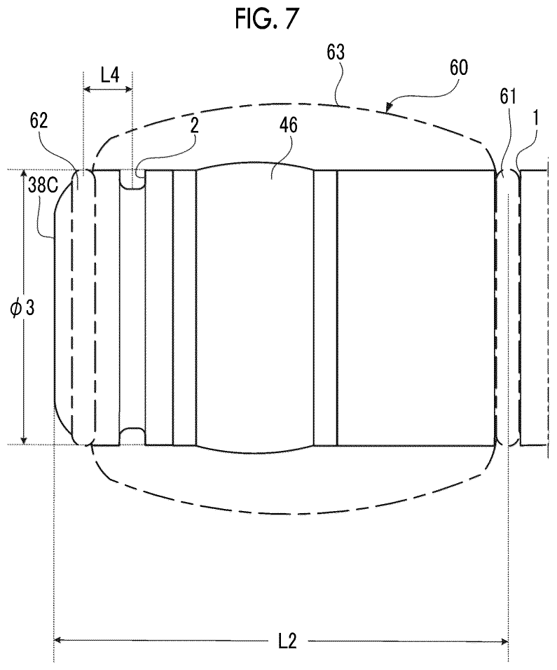

FIG. 7 is a schematic diagram illustrating a process of attaching the balloon 60 to the distal end part 40. The user inserts the distal end part 40 into the opening of the balloon 60 while widening the opening on the bead part 61 side of the balloon 60 by using a jig, and retains the bead part 61 by the proximal end groove 1. FIG. 7 illustrates a diagram in which the work so far is completed.

As described above, in the distal end part 40, the length L2 from the proximal end groove 1 to the distal end surface 38C is longer than the length L1 of the balloon 60 in the axial direction. Therefore, even in the state illustrated in FIG. 7 in which the balloon 60 is pulled to the maximum in the axial direction in a state in which the bead part 61 is retained by the proximal end groove 1, the bead part 62 is located on the proximal end side of the distal end surface 38C as illustrated in FIG. 7.

From the state illustrated in FIG. 7, the user moves the bead part 62 to a position of the distal end groove 2 by pinching the bead part 62 with a finger, and retains the bead part 62 by the distal end groove 2. In a case in which the bead part 62 is moved to the distal end groove 2, the inflatable part 63 may be wound around the bead part 62 as the bead part 62 moves while rotating in the axial direction of the balloon 60, and in this case, the outer diameter of the bead part 62 is increased by the amount of the inflatable part 63 wound around the bead part 62.

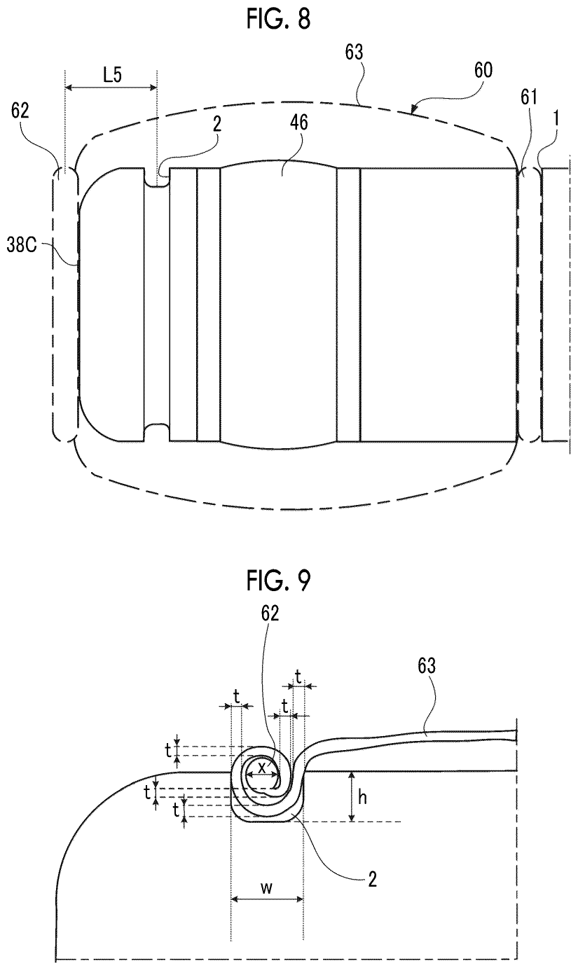

In the ultrasonic endoscope 12, the relationship of length L2>length L1 is satisfied, so that the bead part 62 can be retained at a position close to the distal end groove 2. For example, in a case in which the length L2≤length L1 is satisfied, as illustrated in FIG. 8, in a state in which the bead part 62 is pulled to the maximum in the distal end side, the bead part 62 is located on the distal end side of the distal end surface 38C. In a case in which a maximum distance L4 from the distal end groove 2 to the bead part 62 in a case in which the relationship of length L2>length L1 is satisfied and a maximum distance L5 from the distal end groove 2 to the bead part 62 in a case in which the relationship of length L2≤length L1 is satisfied are compared, the maximum distance L4 is smaller.

As described above, by satisfying the relationship of length L2>length L1, the maximum distance L4 can be reduced, and a movement distance in a case in which the bead part 62 is moved to the distal end groove 2 is shortened. In a case in which the movement distance is short, the inflatable part 63 is less likely to be wound around, and the winding can be prevented. In addition, even in a case in which the winding occurs, the amount of the winding can be reduced.

As described above, by preventing the inflatable part 63 from being wound around the bead part 62, the bead part 62 can be accommodated in the distal end groove 2, and the bead part 62 can be reliably retained by the distal end groove 2. As a result, even in a case in which the inflatable part 63 is inflated, the bead part 62 is prevented from coming off from the distal end groove 2. In addition, even in a case in which the retaining between the distal end groove 2 and the bead part 62 is released, the bead part 62 does not extend to the distal end side of the distal end surface 38C, so that the observation window 78 or the illumination window 80 provided on the distal end surface 38C is not blocked, and the influence on the observation of the endoscopic image can be eliminated.

In addition, in the ultrasonic endoscope 12, the outer diameter φ3 is greater than the inner diameter φ1 of the opening surrounded by the bead part 62 of the balloon 60 and is equal to or less than twice the inner diameter φ1. As described above, since the difference between the outer diameter φ3 and the inner diameter φ1 is not too large, it is not necessary to greatly widen the bead part 62 in a case in which the bead part 62 is moved to the distal end groove 2 from the state of FIG. 7. By moving the bead part 62 without greatly widening the bead part 62, the inflatable part 63 is less likely to be wound around the bead part 62 during the movement. Accordingly, the bead part 62 can be reliably retained by the distal end groove 2.

In addition, in the ultrasonic endoscope 12, the outer diameter φ2 of the distal end surface 38C of the distal end part 40 is less than the inner diameter φ1 of the opening on the bead part 62 side of the balloon 60. Therefore, in the state illustrated in FIG. 7, the opening on the bead part 62 side can be put into a state of being hooked on the curved surface 38B without widening the opening. The curved surface 38B is connected to the distal end groove 2 while being bent, and thus the bead part 62 can move to the distal end groove 2 while being guided by the curved surface 38B. As a result, the bead part 62 is less likely to be wound around the inflatable part 63, and the bead part 62 can be reliably retained by the distal end groove 2.

FIG. 6 illustrates a length L3 from the distal end groove 2 (the center in the longitudinal direction) to the distal end surface 38C. The length L3 corresponds to the movement distance in a case in which the bead part 62 is moved to the distal end groove 2. The shorter the movement distance, the less likely the inflatable part 63 is to be wound around the bead part 62.

It is assumed that the inflatable part 63 is wound around the bead part 62 by one and a half turns. FIG. 9 is a diagram illustrating a state in which the bead part 62 in which the inflatable part 63 is wound one and a half turns is retained by the distal end groove 2. It is found that, in a case in which the inflatable part 63 is wound around the bead part 62 by more than one and a half turns, in consideration of the constraints on the structure (cross-sectional diameter x, thickness t, and material) of the balloon 60 and the structure (depth h and width w) of the proximal end groove 1 and the distal end groove 2, the bead part 62 is likely to come off from the distal end groove 2 in a case in which the balloon 60 is inflated.

As a worst case in which the winding is most likely to occur, in a case in which the cross-sectional diameter x of the bead part 62 is 1.0 mm and the thickness t of the inflatable part 63 is 0.17 mm, the movement distance L of the bead part 62 required for the inflatable part 63 to be wound around the bead part 62 by one and a half turns is L=1.0π+0.5×(1.0+0.17×2)π≈5.25 mm, where π is a circumference ratio. Therefore, in a case in which a certain margin is ensured, an upper limit value of the length L3 is preferably 5.5 mm.

In addition, by forming the balloon 60, the proximal end groove 1, and the distal end groove 2 such that at least one of w≥x+3t or h≥0.5x+2t is satisfied, even in a case in which the inflatable part 63 is wound around the bead part 62 by one and a half turns, the bead part 62 can be prevented from coming off from the distal end groove 2 in a case in which the balloon 60 is inflated. By satisfying the condition of w≥x+3t, as illustrated in FIG. 9, the bead part 62 in which the inflatable part 63 is wound by one and a half turns can be accommodated in the distal end groove 2, and the falling off can be prevented. In addition, by satisfying the condition of h≥0.5x+2t, even in a case in which the inflatable part 63 is wound around the bead part 62 by one and a half turns, half of the bead part 62 can be accommodated in the distal end groove 2, and the falling off can be prevented. Even in a case in which the relationship between the length L2 and the length L1 is not as described above, by forming the balloon 60, the proximal end groove 1, and the distal end groove 2 such that at least one of w≥x+3t or h≥0.5x+2t is satisfied, the falling off of the balloon 60 can be prevented.

In the ultrasonic endoscope 12, the observation window 78 and the illumination window 80 are disposed on the distal end side of the ultrasonic transducer 46, but the present disclosure is not limited to this. For example, the technology of the present disclosure can be applied to a side-viewing ultrasonic endoscope in which the observation window and the illumination window are disposed on the proximal end side of the ultrasonic transducer 46 and the optical axis of the observation window faces the side.

As described above, at least the following matters are set forth in this specification.

(1)

An ultrasonic endoscope comprising: an insertion part that is inserted into a subject; a plurality of oscillators that are disposed on a distal end side of the insertion part in a circumferential direction of the insertion part and that transmit and receive ultrasonic waves; a balloon that is capable of covering the plurality of oscillators; a first groove that is provided on a proximal end side of the plurality of oscillators and that is capable of retaining the balloon; and a second groove that is provided on a distal end side of the plurality of oscillators and that is capable of retaining the balloon, in which a length from the first groove to a distal end of the insertion part is longer than a length of the balloon in an axial direction.

(2)

An ultrasonic endoscope comprising: an insertion part that is inserted into a subject; a plurality of oscillators that are disposed on a distal end side of the insertion part in a circumferential direction of the insertion part and that transmit and receive ultrasonic waves; a balloon that is capable of covering the plurality of oscillators; a first groove that is provided on a proximal end side of the plurality of oscillators and that is capable of retaining the balloon; and a second groove that is provided on a distal end side of the plurality of oscillators and that is capable of retaining the balloon, in which a relationship between a cross-sectional diameter x of an end part of the balloon on a side retained by the second groove, a thickness t of an inflatable part of the balloon, and a width w and a depth h of the second groove satisfies at least one of w≥x+3t or h≥0.5x+2t.

### (3)

The ultrasonic endoscope according to (1) or (2), in which an outer diameter of the insertion part on a distal end side of the second groove is greater than an inner diameter of an opening surrounded by an end part of the balloon on a side retained by the second groove and is equal to or less than twice the inner diameter.

(4)

The ultrasonic endoscope according to any one of (1) to (3), further comprising: a flat surface that is provided at the distal end of the insertion part and that intersects a longitudinal direction of the insertion part, in which an outer diameter of the flat surface is less than an inner diameter of an opening surrounded by an end part of the balloon on a side retained by the second groove.

(5)

The ultrasonic endoscope according to (4), in which the distal end side of the insertion part has a side surface that is located on a proximal end side of the flat surface and on a radially outer side of the insertion part with respect to the flat surface, and a surface that connects the side surface and the flat surface, and as viewed in a radial direction of the insertion part, an angle formed between an imaginary straight line connecting an end of the surface on a flat surface side and an end of the surface on a side surface side and the longitudinal direction is equal to or less than 45°.

(6)

The ultrasonic endoscope according to any one of (1) to (5), further comprising: an illumination window through which illumination light is emitted to the subject, and an observation window through which reflected light of the illumination light is incident, the illumination window and the observation window being provided on the distal end side of the plurality of oscillators in the insertion part.

(7)

The ultrasonic endoscope according to any one of (1) to (6), further comprising: an illumination window through which illumination light is emitted to the subject, and an observation window through which reflected light of the illumination light is incident, the illumination window and the observation window being provided on the distal end side of the insertion part, in which an optical axis of the observation window extends in a longitudinal direction of the insertion part.

(8)

The ultrasonic endoscope according to any one of (1) to (7), in which a material of the balloon is isoprene rubber.

(9)

The ultrasonic endoscope according to any one of (1) to (8), in which a material of the balloon is at least one of a styrene-based elastomer, an olefin-based elastomer, a vinyl chloride-based elastomer, a polyester-based elastomer, a polyurethane-based elastomer, a polyamide-based elastomer, a silicone-based elastomer, or a fluorine-based elastomer.

### EXPLANATION OF REFERENCES

1: proximal end groove
2: distal end groove
3: delivery port
10: ultrasonic endoscope system
12: ultrasonic endoscope
14: ultrasonic processor device
16: endoscopic processor device
18: light source device
20: monitor
21a: water supply tank
21b: suction pump
22: insertion part
24: operating part
26: universal cord
28a: air supply/water supply button
28b: suction button
29: angle knob
30: treatment tool insertion port
32a, 32b, 32c: connector
34a: air supply/water supply tube
34b: suction tube
36: ultrasonic observation part
38: endoscopic observation part
38A: side surface
38B: curved surface
38C: distal end surface
38Bs: imaginary straight line
40: distal end part
41a: distal end component
41b: proximal end side ring
41c: metal ring
42: bendable part
44: soft part
46: ultrasonic transducer
48: oscillator
61, 62: bead part
52: electrode part
52a: individual electrode
52b: common electrode
54: backing material layer
56: flexible printed substrate
58: coaxial cable
60: balloon
63: inflatable part
64: acoustic matching layer
66: acoustic lens
72: signal line bundle
76: treatment tool outlet port
78: observation window
80: illumination window
82: washing nozzle
85: observation system unit
86: objective lens
88: prism
90: imaging element
92: substrate
94: signal cable
98: light guide
100: air supply/water supply channel
L1, L2, L3: length
L4, L5: maximum distance

What is claimed is:

1. An ultrasonic endoscope comprising:
an insertion part that is to be inserted into a subject;
a plurality of oscillators that are disposed on a distal end side of the insertion part in a circumferential direction of the insertion part and that transmit and receive ultrasonic waves;
a balloon that is capable of covering the plurality of oscillators;
a first groove that is provided on a proximal end side of the plurality of oscillators and that is capable of retaining the balloon; and
a second groove that is provided on a distal end side of the plurality of oscillators and that is capable of retaining the balloon,
wherein a length from the first groove to a distal end of the insertion part is longer than a length of the balloon in an axial direction.

2. An ultrasonic endoscope comprising:
an insertion part that is to be inserted into a subject;
a plurality of oscillators that are disposed on a distal end side of the insertion part in a circumferential direction of the insertion part and that transmit and receive ultrasonic waves;
a balloon that is capable of covering the plurality of oscillators;
a first groove that is provided on a proximal end side of the plurality of oscillators and that is capable of retaining the balloon; and
a second groove that is provided on a distal end side of the plurality of oscillators and that is capable of retaining the balloon,
wherein a relationship between a cross-sectional diameter x of an end part of the balloon on a side retained by the second groove, a thickness t of an inflatable part of the balloon, and a width w and a depth h of the second groove satisfies at least one of:
w≥x+3t; or
h≥0.5x+2t.

3. The ultrasonic endoscope according to claim 1,
wherein an outer diameter of the insertion part on a distal end side of the second groove is greater than an inner diameter of an opening surrounded by an end part of the balloon on a side retained by the second groove and is equal to or less than twice the inner diameter.

4. The ultrasonic endoscope according to claim 2,
wherein an outer diameter of the insertion part on a distal end side of the second groove is greater than an inner diameter of an opening surrounded by an end part of the balloon on a side retained by the second groove and is equal to or less than twice the inner diameter.

5. The ultrasonic endoscope according to claim 1, further comprising:
a flat surface that is provided at the distal end of the insertion part and that intersects a longitudinal direction of the insertion part,
wherein an outer diameter of the flat surface is less than an inner diameter of an opening surrounded by an end part of the balloon on a side retained by the second groove.

6. The ultrasonic endoscope according to claim 2, further comprising:
a flat surface that is provided at the distal end of the insertion part and that intersects a longitudinal direction of the insertion part,
wherein an outer diameter of the flat surface is less than an inner diameter of an opening surrounded by an end part of the balloon on a side retained by the second groove.

7. The ultrasonic endoscope according to claim 5,
wherein the distal end side of the insertion part has a side surface that is located on a proximal end side of the flat surface and on a radially outer side of the insertion part with respect to the flat surface, and a surface that connects the side surface and the flat surface, and
as viewed in a radial direction of the insertion part, an angle formed between an imaginary straight line connecting an end of the surface on a flat surface side and an end of the surface on a side surface side and the longitudinal direction is equal to or less than 45°.

8. The ultrasonic endoscope according to claim 6,
wherein the distal end side of the insertion part has a side surface that is located on a proximal end side of the flat surface and on a radially outer side of the insertion part with respect to the flat surface, and a surface that connects the side surface and the flat surface, and
as viewed in a radial direction of the insertion part, an angle formed between an imaginary straight line connecting an end of the surface on a flat surface side and an end of the surface on a side surface side and the longitudinal direction is equal to or less than 45°.

9. The ultrasonic endoscope according to claim 1, further comprising:
an illumination window through which illumination light is emitted to the subject, and an observation window through which reflected light of the illumination light is incident, the illumination window and the observation window being provided on the distal end side of the plurality of oscillators in the insertion part.

10. The ultrasonic endoscope according to claim 2, further comprising:
an illumination window through which illumination light is emitted to the subject, and an observation window through which reflected light of the illumination light is incident, the illumination window and the observation window being provided on the distal end side of the plurality of oscillators in the insertion part.

11. The ultrasonic endoscope according to claim 1, further comprising:
an illumination window through which illumination light is emitted to the subject, and an observation window through which reflected light of the illumination light is incident, the illumination window and the observation window being provided on the distal end side of the insertion part,
wherein an optical axis of the observation window extends in a longitudinal direction of the insertion part.

12. The ultrasonic endoscope according to claim 2, further comprising:
an illumination window through which illumination light is emitted to the subject, and an observation window through which reflected light of the illumination light is incident, the illumination window and the observation window being provided on the distal end side of the insertion part,
wherein an optical axis of the observation window extends in a longitudinal direction of the insertion part.

13. The ultrasonic endoscope according to claim 1,
wherein a material of the balloon is isoprene rubber.

14. The ultrasonic endoscope according to claim 2,
wherein a material of the balloon is isoprene rubber.

15. The ultrasonic endoscope according to claim 1,
wherein a material of the balloon is at least one of a styrene-based elastomer, an olefin-based elastomer, a vinyl chloride-based elastomer, a polyester-based elastomer, a polyurethane-based elastomer, a polyamide-based elastomer, a silicone-based elastomer, or a fluorine-based elastomer.

16. The ultrasonic endoscope according to claim 2,
wherein a material of the balloon is at least one of a styrene-based elastomer, an olefin-based elastomer, a vinyl chloride-based elastomer, a polyester-based elastomer, a polyurethane-based elastomer, a polyamide-based elastomer, a silicone-based elastomer, or a fluorine-based elastomer.
